(19) United States
(12) Patent Application Publication
(10) Pub. No.: US 2019/0175054 A1
(43) Pub. Date: Jun. 13, 2019

(54) MULTI-LAYER COMPLIANT FORCE OR PRESSURE SENSING SYSTEM APPLICABLE FOR ROBOTIC SENSING AND ANATOMICAL MEASUREMENTS

(71) Applicants: University of Maryland, College Park, MD (US); University of Illinois at Urbana-Champaign, Urbana, IL (US)

(72) Inventors: Elisabeth Smela, Silver Spring, MD (US); Miao Yu, Potomac, MD (US); Hugh A. Bruck, Wheaton, MD (US); Ying Chen, College Park, MD (US); Thenkurussi Kesavadas, Mahomet, IL (US)

(73) Assignees: University of Maryland, College Park, MD (US); The Board of Trustees of the University of Illinois, Urbana, IL (US)

(21) Appl. No.: 16/112,609

(22) Filed: Aug. 24, 2018

Related U.S. Application Data
(60) Provisional application No. 62/549,672, filed on Aug. 24, 2017.

(51) Int. Cl. A61B 5/053; A61B 5/00

(52) U.S. Cl. CPC A61B 5/0536; A61B 5/0002; G01N 19/00; A61B 5/6804; A61B 5/4387; A61B 5/4312

(57) ABSTRACT

A pressure sensing system includes at least two pressure sensing layers. The first pressure sensing layer includes a first sensing system configured in a layer, a first layer of foam having a Young's modulus and mounted between a first sensing system configured in a layer, and a second sensing system configured in a layer; at least a second pressure sensing layer including the second sensing system configured in a layer, and a second layer of foam having a Young's modulus that is greater than the Young's modulus of the first layer of foam and mounted between the second sensing system configured in a layer and a rigid substrate having a Young's modulus greater than the layer of the first sensing system, the first layer of foam, the layer of the second sensing system, and the second layer of foam. The pressure sensing system thereby defines a multi-layer pressure sensing system.

### CROSS-REFERENCE TO RELATED APPLICATIONS

This application claims the benefit of, and priority to, U.S. Provisional Patent Application No. 62/549,672 filed on Aug. 24, 2017, entitled “Tactile Sensing Palpation Bra for Breast Cancer Diagnosis” by Elisabeth Smela et al., the entire contents of which are incorporated herein by reference.

### STATEMENT OF GOVERNMENT SUPPORT

This invention was made with U.S. government support under IIS1317913 awarded by NSF. The U.S. government has certain rights in the invention.

### BACKGROUND

### 1. Technical Field

The present disclosure relates to pressure sensing systems and more particularly pressure sensing systems that include, but are not limited to, applications for robotic sensing.

### 2. Discussion of Related Art

Health care providers can use touch to determine the size, texture, and location of a tumor. As part of a clinical breast examination (CBE) to screen for breast cancer, a physician or other trained health practitioner performs a manual palpation. Typically, varying pressure is applied using the pads of three fingers in circular motions, in a systematic pattern to cover the entire breast. Palpation can detect malignant masses because they are generally harder than the surrounding tissue and are often fixed to surrounding skin and soft tissue.

Other types of cancer (e.g., of the throat and tongue) can be detected similarly.

FIG. 1 illustrates a female P1 performing a self-examination by palpation which is feeling the breasts with the fingers or hands during a physical examination.

FIG. 2 illustrates conventional mammography 10 of a patient P2 which is a radiological examination of the breast, and is used to screen for or evaluate tumors and other abnormalities and is utilized in geographical locations having adequate resources. The inset shows a detected abnormality 15.

However, in parts of the world without medical personnel who are properly trained, and without the benefit of conventional mammography, breast cancer often goes undetected.

### SUMMARY

The embodiments of the present disclosure provide significant and non-obvious advantages over the prior art by providing a pressure sensing system including: at least two pressure sensing layers, the first pressure sensing layer of the at least two pressure sensing layers including: a first sensing system configured in a layer; and a first layer of foam having a Young's Modulus and mounted between the first sensing system configured in a layer and a second sensing system configured in a layer; and at least a second pressure sensing layer of the at least two pressure sensing layers including: the second sensing system configured in a layer; and a second layer of foam having a Young's modulus that is greater than the Young's modulus of the first layer of foam and mounted between the second sensing system configured in a layer and a rigid substrate having a Young's modulus greater than the layer of the first sensing system, the first layer of foam, the layer of the second sensing system, and the second layer of foam, the at least two pressure sensing layers defining thereby a multi-layer pressure sensing system.

In an embodiment, the pressure sensing system may be configured as a tumor detection system. The tumor detection system includes an anatomical contact material configured to contact or apply pressure to at least one anatomical mass that extends from the body of a user of the system or to a body surface of a user of the system. The anatomical mass includes an outer surface with respect to the body of the user of the device. The anatomical contact material includes an interior surface and an exterior surface with respect to the outer surface of the at least one anatomical mass or to the body surface. The multi-layer pressure sensing system includes an interior surface and an exterior surface with respect to the outer surface of the at least one anatomical mass or to the body surface. The interior surface of the multi-layer pressure sensing system is configured to be positioned over the outer surface of the at least one anatomical mass, or body surface, between the at least one anatomical mass, or body surface, and the interior surface of the anatomical contact material. The rigid substrate of the multi-layer pressure sensing system may be configured as a flexible insufflation reservoir including an interior surface and an exterior surface with respect to the outer surface of the at least one anatomical mass or the body surface. The flexible insufflation reservoir may be configured wherein the interior surface of the flexible insufflation reservoir can be positioned over the exterior surface of the multi-layer pressure sensing system and wherein the interior surface of the anatomical contact material can be positioned over the exterior surface of the flexible insufflation reservoir, wherein inflation of the flexible insufflation reservoir causes pressure to be applied to the multi-layer pressure sensing system and to the at least one anatomical mass, or body surface, to enable detection of a tumor within the at least one anatomical mass, or body surface, by the multi-layer pressure sensing system.

The multi-layer pressure sensing system may include an electrical impedance tomography circuit. The electrical impedance tomography circuit may include a plurality of pairs of adjacent electrodes, wherein current is injected into an adjacent pair of electrodes such that voltage readings obtained from the remaining pairs of the plurality of pairs of adjacent electrodes enable reconstruction of an image from the measured voltage readings.

The electrical impedance tomography circuit may include circuitry enabling wireless transmission of data readings from the multi-layer pressure sensing system to a remote receiver location.

The multi-layer pressure sensing system may include an array of strip sensors disposed over a layer of foam padding.

The anatomical support or contact material configured to contact or apply pressure to at least one anatomical mass that extends from the body of a user, or a body surface of a user, may be configured as a brassiere to support the breasts of a user to detect tumors occurring within at least one breast of the user.

The anatomical support or contact material configured to contact or apply pressure to at least one anatomical mass that extends from the body of a user, or a body surface of the user, may be configured as a male athletic supporter to support the testicles of a male user to detect tumors occurring within at least one testicle of the male user.

### BRIEF DESCRIPTION OF THE DRAWINGS

The above-mentioned advantages and other advantages will become more apparent from the following detailed description of the various exemplary embodiments of the present disclosure with reference to the drawings wherein:

FIG. 1 illustrates a female performing a self-examination by palpation which is feeling the breasts with the fingers or hands during a physical examination;

FIG. 2 illustrates conventional mammography of a patient which is a radiological examination of the breast, and is used to screen for or evaluate tumors and other abnormalities and is utilized in geographical locations having adequate resources and wherein the inset shows a detected abnormality;

FIG. 3 illustrates a portion of a multi-layer force or pressure sensing system that is configured to increase sensitivity of pressure or force measurements to detect a mass embedded in a tissue or a substance or material wherein the mass has a density greater than the density of the tissue or substance or material;

FIG. 4A illustrates the multi-layer force or pressure sensing system of FIG. 1.1 under a uniform pressure or force (indicated by the arrows) applied directly to a surface of a first sensing skin layer wherein the multi-layer sensing system includes a layer of foam having a first Young's Modulus mounted between layered first sensing system and a layered second sensing system and further includes a second layer of foam having a Young's modulus that is greater than the Young's modulus of the first layer of foam and is mounted between the layered second sensing system and a backing material or rigid substrate;

FIG. 4B illustrates the uniform pressure or force applied to a uniform layer of tissue mounted on the first sensing skin layer of the multi-layer force or pressure sensing system or material of FIG. 4A;

FIG. 4C illustrates the multi-layer force or pressure sensing system of FIG. 1.1(b) wherein a hard mass is embedded in the uniform layer of tissue and wherein the uniform pressure is applied to the uniform layer of tissue;

FIG. 4D illustrates the multi-layer force or pressure sensing system of FIG. 4C wherein a uniform pressure greater than the uniform pressure applied in FIG. 4C is applied to uniform layer of tissue in which the hard mass is embedded such that a portion of the layered first sensing system converges with a portion of the layered second sensing system;

FIG. 5A illustrates a schematic representation of the signals from two sensing layers upon increasing the applied force (pressure) linearly over time as a ramp function wherein the first sensing layer responds earlier than the second sensing layer;

FIG. 5B is a schematic representation of possible signals from the sensing layer upon increasing the applied pressure wherein larger tumor lumps may be detected earlier and softer tumor lumps may have a different slope;

FIG. 6A illustrates an embodiment of the pressure sensing system configured as a tumor detection system that includes an anatomical contact material configured to contact or apply pressure to at least one anatomical mass or body surface, e.g., breasts cups, that are in contact with the breast tissue surface;

FIG. 6B illustrates the pressure distribution in the breast in the presence of a hard mass in the breast tissue utilizing the palpation brassiere tumor detecting system of FIG. 6A;

FIG. 7A is a cross-sectional view of the multi-layer pressure sensing system as applied to tumor detection as shown in FIGS. 6A and 6B;

FIG. 7B illustrates representative tumor masses wherein the sensing sheet is in electrical communication with a portable electronic system;

FIG. 7C illustrates the tumor detection system;

FIG. 8 illustrates a distributed sensing system wherein electrical impedance tomography (EIT) is utilized to image a continuous sensor area;

FIG. 9 illustrates the sensor in a distributed system which now includes multiplexers wherein analog input measurements are transmitted to a data acquisition card (DAQ) where the analog input measurements are converted to digital output;

FIG. 9A1 illustrates two loading points for a mechanical sensor diameter of 10 cm where the electrical reading images are shown as dark spots in FIG. 9A2;

FIG. 9B1 illustrates a thermal sensor having a square outline boundary and wherein thermal sensing readings are shown as a quadrilateral image in FIG. 9B2;

FIG. 10A illustrates a detailed view of the electrical sensor with electrodes attached at the periphery for EIT and resting on a compressible substrate;

FIG. 10B illustrates the corresponding EIT image showing the dark areas representing tumor locations;

FIG. 11A illustrates an array strip sensor that is formed of a series of orthogonally positioned crossing strips of eight (8) rows and eight (8) columns;

FIG. 11B illustrates the corresponding image in response to a touch at row 4, column 4;

FIG. 12A illustrates the pressure sensing system configured as a tumor detection system as described above with respect to FIGS. 7A-7C but as a phantom for testing;

FIG. 12B illustrates a portable electronics system that is in electrical communication with the tumor detection system;

FIG. 13A illustrates conductivity images converted to mm Hg of phantoms (a) with no lumps; (b) with one (1) lump; and (c) with two (2) lumps;

FIG. 13B illustrates a contour plot of the two-lump 80 mm Hg image; and

FIG. 14 is a schematic diagram for a method of manufacturing the piezoelectric exfoliated graphite (EG)/latex sensing layer.

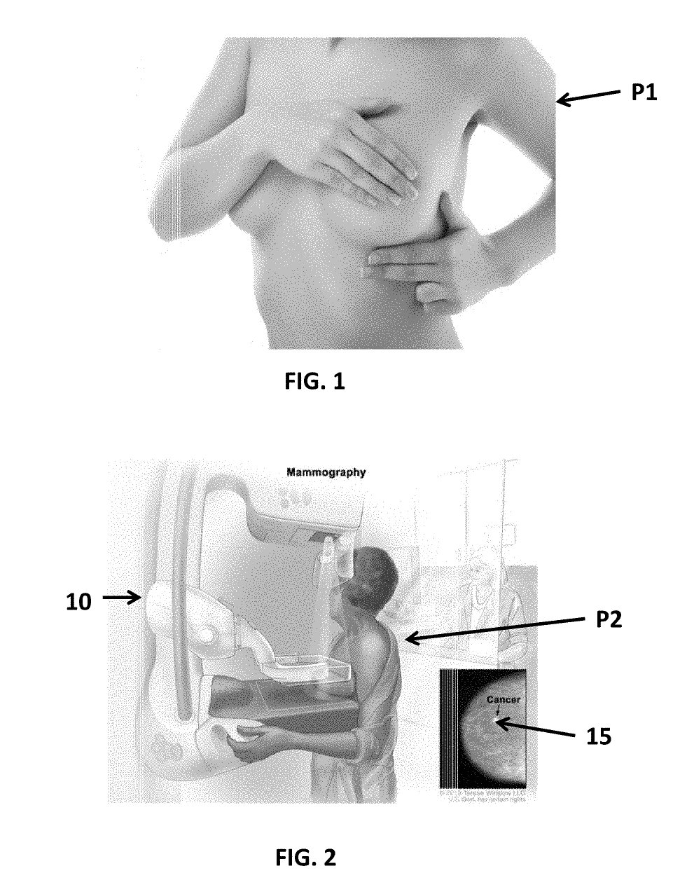

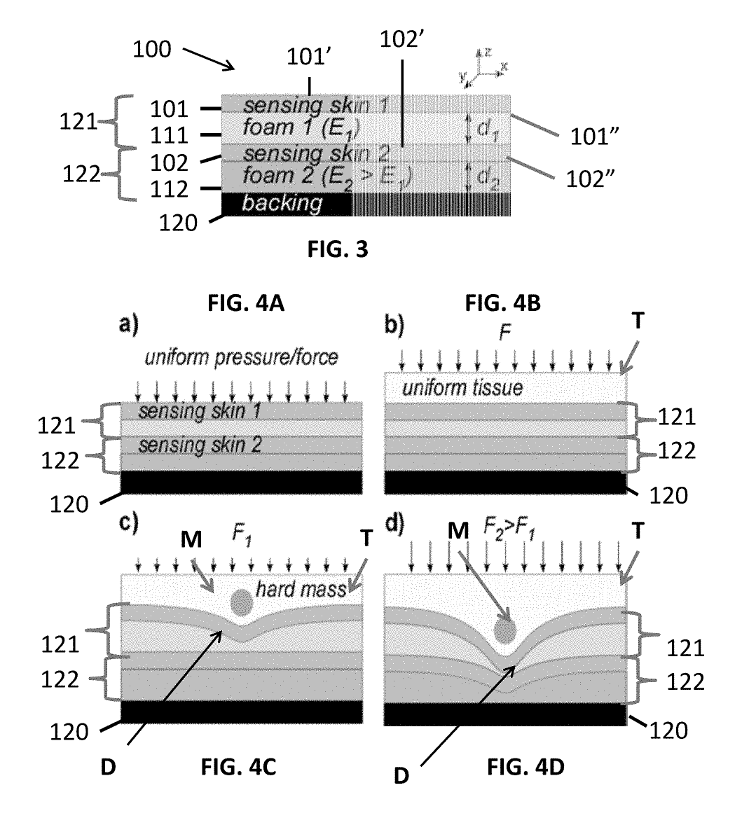

### DETAILED DESCRIPTION

For the purposes of promoting an understanding of the principles of the present disclosure, reference will now be made to the exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The word “exemplary” is used herein to mean “serving as an example, instance, or illustration.” Any embodiment described herein as “exemplary” is not necessarily to be construed as preferred or advantageous over other embodiments.

It is to be understood that the method steps described herein need not necessarily be performed in the order as described. Further, words such as “thereafter,” “then,” “next,” etc., are not intended to limit the order of the steps. Such words are simply used to guide the reader through the description of the method steps.

The implementations described herein may be implemented in, for example, a method or a process, an apparatus, a software program, a data stream, or a signal. Even if only discussed in the context of a single form of implementation (for example, discussed only as a method), the implementation of features discussed may also be implemented in other forms (for example, an apparatus or program). An apparatus may be implemented in, for example, appropriate hardware, software, and firmware. The methods may be implemented in, for example, an apparatus such as, for example, a processor, which refers to processing devices in general, including, for example, a computer, a microprocessor, an integrated circuit, or a programmable logic device. Processors also include communication devices, such as, for example, computers, cell phones, tablets, portable/personal digital assistants, and other devices that facilitate communication of information between end-users within a network.

The general features and aspects of the present disclosure remain generally consistent regardless of the particular purpose. Further, the features and aspects of the present disclosure may be implemented in system in any suitable fashion, e.g., via the hardware and software configuration of system or using any other suitable software, firmware, and/or hardware. For instance, when implemented via executable instructions, such as the set of instructions, various elements of the present disclosure are in essence the code defining the operations of such various elements. The executable instructions or code may be obtained from a computer-readable medium (e.g., a hard drive media, optical media, EPROM, EEPROM, tape media, cartridge media, flash memory, ROM, memory stick, and/or the like) or communicated via a data signal from a communication medium (e.g., the Internet). In fact, readable media may include any medium that may store or transfer information.

The present disclosure relates to a multi-layer tactile or pressure-sensing system as described in “Characterization of a compliant multi-layer system for tactile sensing with enhanced sensitivity and range” by Ying Chen et al., Smart Materials and Structures, published on May 3, 2018 [(Smart Mater. Struct. 27 (2018) 065005 (15 pp); https://doi.org/10.1088/1361-665X/aabc29], the entire contents of which are hereby incorporated by reference herein.

A multi-layer tactile sensing system, according to the present disclosure includes alternating layers of sensing “skin” and padding, with the padding increasing in stiffness further from the top surface. The sensing “skin” comprises a piezoresistive thin film on a stretchable substrate. Piezoresistors change electrical resistance when they are stretched. A composite of exfoliated graphite (EG) mixed into latex as the piezoresistive material is utilized because it is stretchable and can be painted onto a wide variety of surfaces as a thin film. Electrical leads are attached to the sensing skin to allow the resistance of the piezoresistor to be monitored. Latex sheet or fabric is utilized for the stretchable substrate. For the padding, foam is employed, but other materials, such as silicone elastomers, can also be used. The layers are supported on a backing that does not stretch.

As defined herein, a rigid substrate is a material having the largest Young's modulus E or stiffness value as compared to the other materials utilized in the multi-layer tactile or pressure sensing system.

Pressure is detected by a tactile sensing material that includes alternating layers of sensing skin and padding with electrodes attached to the periphery of the said sensing material. Sensing skin includes piezoresistive thin film on a stretchable substrate. Padding can be foam but other materials such as elastomers can also be used. Padding layers increase in stiffness further from the top surface. Piezoresistors change electrical resistance when they are stretched.

In an embodiment, the present disclosure relates to an automated device for breast palpation for the detection of breast tumors that are stiffer than the surrounding tissue. The device comprises both hardware and software and includes a continuous sensor to quantitatively image cancerous lumps, which are stiffer than healthy tissue. This automated palpation system mimics a clinical breast exam, without requiring a healthcare professional.

The sensor is compliant and conforms to the breast, enabling imaging of stiff inclusions.

The system includes a piezoresistive sensing sheet and an inflatable balloon or insufflation reservoir built into a fabric brassiere, along with a portable electronic system.

As is known in the art, Piezoresistivity is a change in electrical resistance under strain or external force.

The sensor according to the present disclosure includes conductive carbon nanoparticles embedded in latex, which is painted onto a rubber sheet. When this material is stretched, the carbon particles become separated, losing electrical connection with each other and causing the resistance to increase.

Electrical impedance tomography (EIT) is an imaging technology used in the medical field with optical signals.

For electrical resistance mapping, EIT is performed by injecting current into pairs of equidistantly-placed electrodes on the periphery of a continuous resistive area and recording the voltages at all the other electrodes.

The resistance over the entire area is reconstructed from these voltages.

Pressure is detected by tactile sensing material consisting of alternating layers of sensing skin and padding with electrodes attached to the periphery of the said sensing material. Sensing skin comprises of piezoresistive thin film on a stretchable substrate. Padding can be foam but other materials such as elastomers can also be used. Padding layers increase in stiffness further from the top surface. Piezoresistors change electrical resistance when they are stretched.

To detect pressure difference on the soft tissue of, for example, a breast, an inflation membrane and pressurization system are required to press the sensing material against the breast to detect pressure differences caused by the presence of the malignant tissue.

Two embodiments of the cancer tissue or two detection method include: 1) one continuous piece of sensing material and 2) array of of sensing material strips “weaved” through.

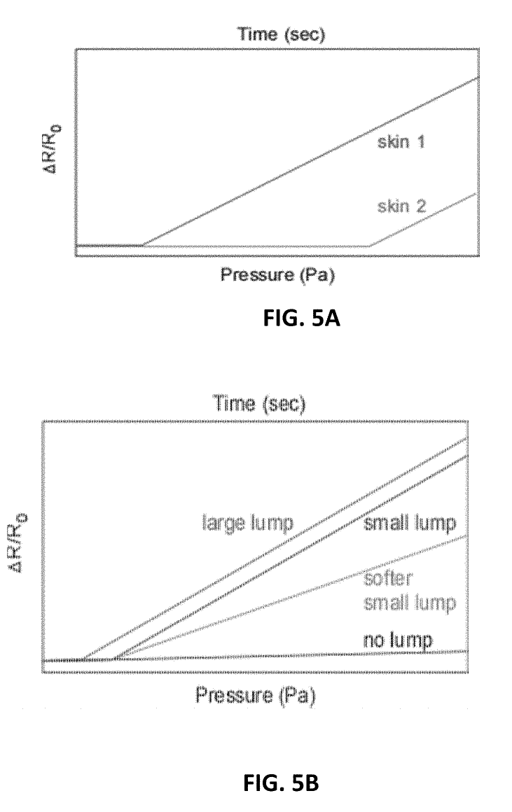

FIG. 3 illustrates a portion of a multi-layer force or pressure sensing system 100 that is configured to increase sensitivity of pressure or force measurements to detect a mass embedded in a tissue or a substance or material wherein the mass has a density greater than the density of the tissue or substance or material.

Multi-layer tactile sensing is illustrated here for two layers. The sensing “skin” consists of a piezoresistive thin film on a stretchable material, such as a latex membrane or a fabric. The Young's modulus (stiffness), E, of the padding foam is lower closer to the surface.

More particularly, the pressure sensing system 100 includes at least two pressure sensing layers 121 and 122. The first pressure sensing layer 121 includes a first sensing system 101 configured in a layer; and a layer of foam 111 having a Young's Modulus and mounted between first sensing system 101 configured in a layer and a second sensing system 102 configured in a layer.

At least a second pressure sensing layer 122 includes the second sensing system 102 configured in a layer; and a second layer of foam 122 having a Young's modulus that is greater than the Young's modulus of the first layer of foam 121 and mounted between the second sensing system 102 configured in a layer and a rigid substrate 120 such that the at least two pressure sensing layers 121 and 122 define thereby a multi-layer pressure sensing system (the pressure sensing system 100).

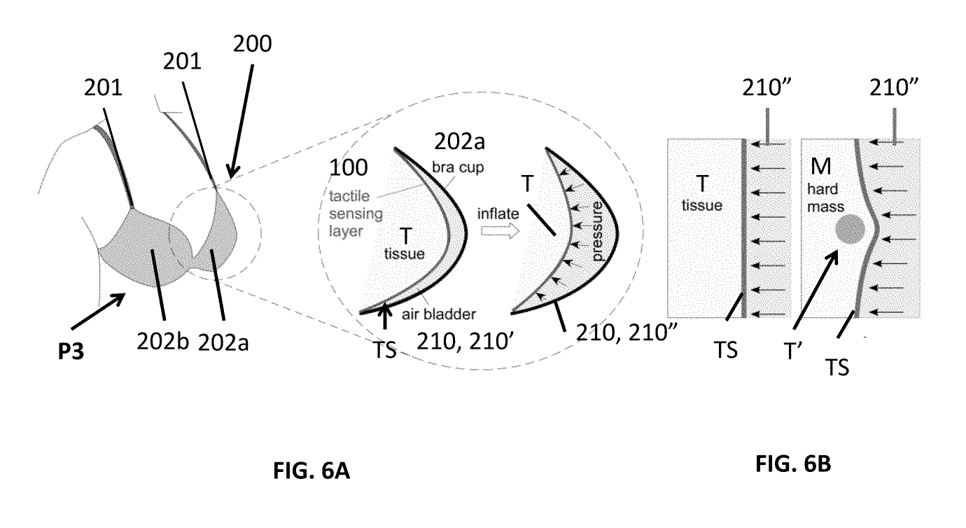

FIG. 4A illustrates the multi-layer force or pressure sensing system 100 of FIG. 3 under a uniform pressure or force (indicated by the arrows) applied directly to a surface 101′ of first sensing skin layer 101 wherein the multi-layer sensing system 100 includes a layer of foam 111 having a first Young's Modulus E1 mounted between layered first sensing system 101 and layered second sensing system 102 and further includes a second layer of foam 112 having a Young's modulus E2 that is greater than the Young's modulus E1 of the first layer of foam 111 and is mounted between the layered second sensing system 122 and a backing material or rigid substrate 120.

FIG. 4B illustrates the uniform pressure or force applied to a uniform layer of tissue T mounted on the first sensing skin layer 101 of the multi-layer force or pressure sensing system or material 100 of FIG. 4A.

FIG. 4C illustrates the multi-layer force or pressure sensing system 100 of FIG. 4B wherein a hard mass M is embedded in the uniform layer of tissue T and wherein the uniform pressure F is applied to the uniform layer of tissue T.

FIG. 4D illustrates the multi-layer force or pressure sensing system 100 of FIG. 4C wherein a uniform pressure F2 greater than the uniform pressure F1 applied in FIG. 4C is applied to uniform layer of tissue T in which the hard mass M is embedded such that a portion of the layered first sensing system 121 converges with a portion of the layered second sensing system 122.

In FIG. 4A: The sensors are not stretched under pressure that compresses the foam padding uniformly.

In FIG. 4B: A uniform overlying layer, for example of tissue, under uniform pressure will also just compress the padding uniformly.

In FIG. 4C: Under a small force a hard mass within the tissue will result in local deformation of the first layer of padding, and thus a stretching of the upper layer sensing skin, resulting in a change in resistance.

In FIG. 4D: For a greater force, the second layer of foam will also be indented, resulting in a signal from the second sensing layer also.

The sensing ‘skin’ is composed of a piezoresistive thin film on a stretchable material, such as a latex membrane or a fabric.

The multi-layer sensing system is composed of two layer piezoresistive sensing skins padded with two-layer material with distinct stiffness.

The layers are supported on a backing that does not stretch.

The rigid substrate is a final layer that does not significantly stretch wherein the Young's modulus of the rigid substrate is greater than that of the other layers.

The multi-layer pressure sensing systems as configured produces a novel working system for breast cancer detection.

The multilayered pressure sensing material is thus comprised of alternating layers of piezoresistive sensing skin and padding foam or elastomers of varying stiffness packed by unstretchable backing.

The multi-layer pressure sensing system thus provides a simple compliant sensing structure over a large area with a larger dynamic range as compared to the prior art.

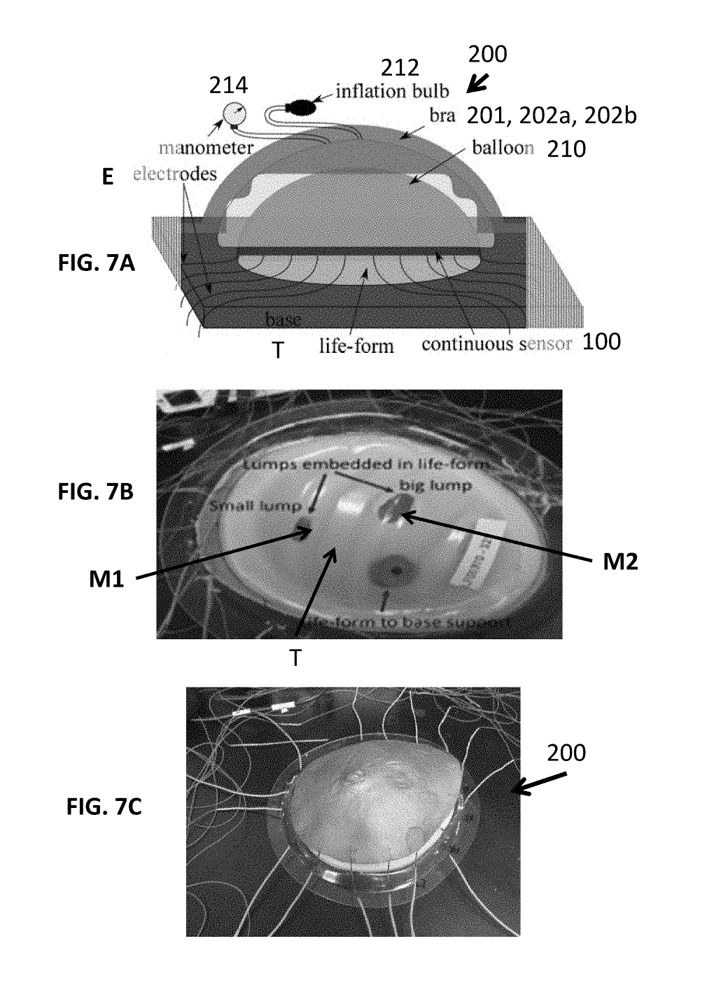

FIG. 5A illustrates a schematic representation of the signals from two sensing layers upon increasing the applied force (pressure) linearly over time as a ramp function wherein the first sensing layer responds earlier than the second sensing layer.

FIG. 5B is a schematic representation of possible signals from the sensing layer upon increasing the applied pressure wherein larger tumor lumps may be detected earlier and softer tumor lumps may have a different slope.

As described in more detail below, the multi-layer pressure sensing system may include an electrical impedance tomography circuit.

The electrical impedance tomography circuit includes a plurality of pairs of adjacent electrodes wherein current is injected into an adjacent pair of electrodes such that voltage readings obtained from the remaining pairs of the plurality of pairs of adjacent electrodes enable reconstruction of an image from the measured voltage readings.

The electrical impedance tomography circuit may include circuitry enabling wireless transmission of data readings from the multi-layer pressure sensing system to a remote receiver location, or may include hard-wired or other types of data transmission methods.

As described further below, the multi-layer pressure sensing system may include as an alternative an array of strip sensors disposed over a layer of foam padding.

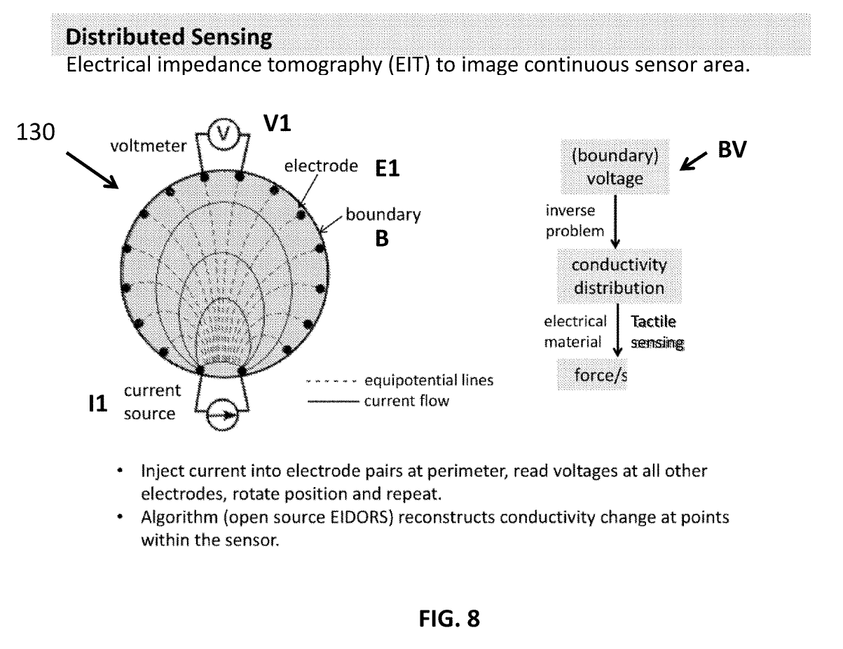

FIG. 6A illustrates an embodiment of the pressure sensing system 100 configured as a tumor detection system 200 that includes an anatomical contact material 201 configured to contact or apply pressure to at least one anatomical mass or body surface, e.g., breasts cups 202a and 202b that are in contact with the breast tissue surface TS of a patient P3. An insufflation reservoir 210 is positioned in the non-inflated configuration 210′ and then inflated to the inflated configuration 210″.

FIG. 6B illustrates the pressure distribution in the breast in the presence of a hard mass in the breast tissue utilizing the palpation brassiere tumor detecting system of FIG. 6A. A hard mass M is shown as appearing under a tissue deformation area T′ as pressure from the insufflation reservoir 210 is applied.

The system may be applied to non-anatomical masses and at least to anatomical masses in general, i.e. not just those which extend from the body, for example, measuring for lumps in the abdomen or on a limb.

The system 100 is thus also capable of detecting masses containing other biological or elemental materials beyond the definition of “tumor”.

The anatomical support material includes an interior surface and an exterior surface with respect to the outer surface of the at least one anatomical mass. The multi-layer pressure sensing system includes an interior surface and an exterior surface with respect to the outer surface of the at least one anatomical mass. The interior surface of the multi-layer pressure sensing system is configured to be positioned over the outer surface of the at least one anatomical mass between the at least one anatomical mass and the interior surface of the anatomical support material.

The rigid substrate of the multi-layer pressure sensing system is configured as the flexible insufflation reservoir 210 that includes an interior surface and an exterior surface with respect to the outer surface of the at least one anatomical mass,

The flexible insufflation reservoir is configured wherein the interior surface of the flexible insufflation reservoir can be positioned over the exterior surface of the multi-layer pressure sensing system and wherein the interior surface of the anatomical support material can be positioned over the exterior surface of the flexible insufflation reservoir,

Inflation of the flexible insufflation reservoir causes pressure to be applied to the multi-layer pressure sensing system and to the at least one anatomical mass to enable detection of a tumor or other anatomical structure within the at least one anatomical mass or body surface by the multi-layer pressure sensing system.

The increasing pressure as the bladder inflates provides a time-dependent signal whose slope and origin contain information about the tissue composition. Applying EIT or a sensor array furnishes additional spatial information.

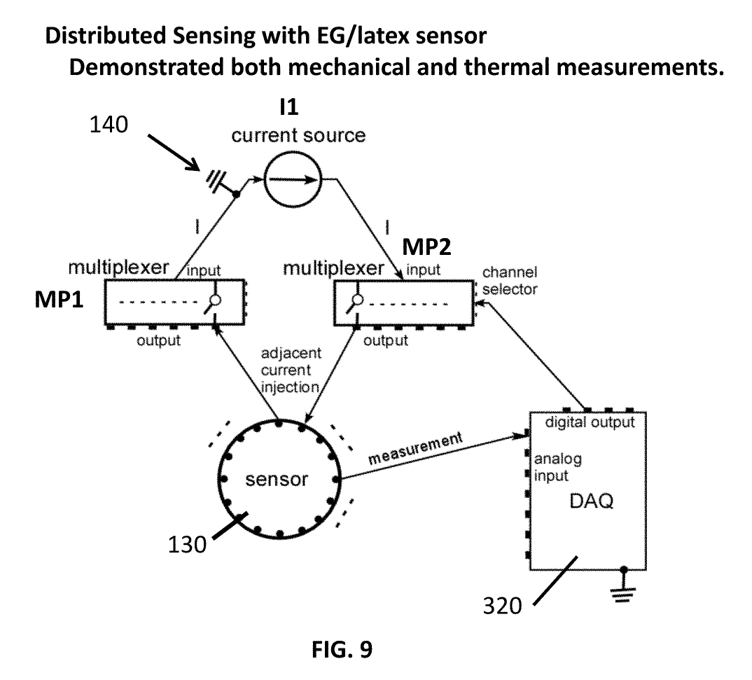

FIG. 7A illustrates a detailed cross-section of a continuous sensor to quantitatively image cancerous lumps, which are stiffer than healthy tissue. This automated palpation system mimics a clinical breast exam, without requiring a healthcare professional.

The sensor is compliant and conforms to the breast, enabling imaging of stiff inclusions.

The system is envisioned to consist of a piezoresistive sensing sheet and an inflatable balloon built into a fabric bra, along with a portable electronic system.

More particularly, FIG. 7A is a cross-sectional view of the multi-layer pressure sensing system as applied to tumor detection as shown in FIGS. 6A and 6B. The tumor detection system 200 includes a piezoelectric sensing sheet and an insufflation reservoir 210 in the form of an inflatable balloon having an inflation bulb 212 and a manometer 214 over a life-form representing tissue T wherein electrodes E are in electrical communication with a portable electronic system.

FIG. 7B illustrates representative tumor masses M1 and M2 wherein the sensing sheet is in electrical communication with a portable electronic system.

FIG. 7C illustrates the tumor detection system 200.

The anatomical support material configured to support at least one anatomical mass that extends from the body of a user of the device is configured as a brassiere to contact the breasts of a female user to detect tumors occurring within at least one breast of the female user or as a piece of material to detect tumors in male breasts as well.

The tumor detection system may include wherein the anatomical support or contact material is configured to contact or apply pressure to at least one anatomical mass that extends from the body of a user of the device is configured as a male athletic supporter to support the testicles of a male user to detect tumors occurring within at least one testicle of the male user.

As indicated above, additional body surfaces and conditions besides tumors may be measured such as the limbs or torso, whether in males or females.

The system may be configured as a brassiere/athletic supporter wherein the device contacts the breasts/testicles to detect internal masses.

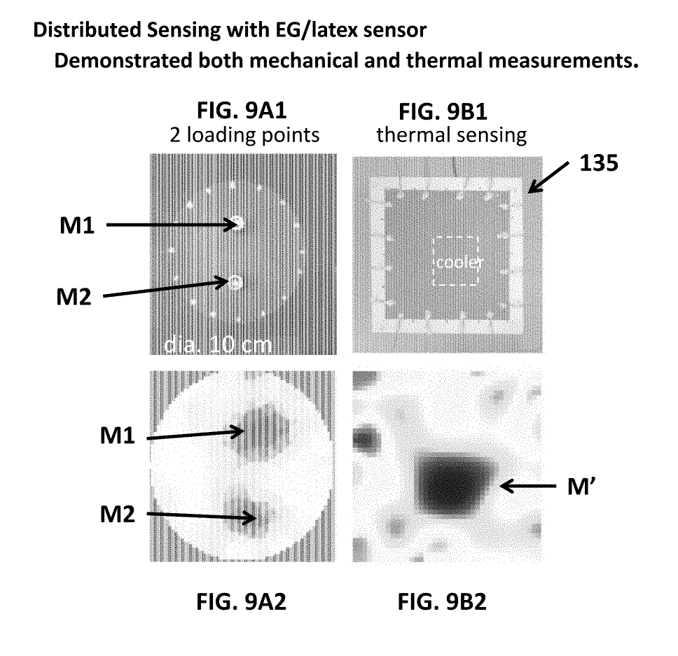

FIG. 8 illustrates a distributed sensing system 130 wherein electrical impedance tomography (EIT) is utilized to image a continuous sensor area. The system includes a voltmeter V1, current source I1, electrodes E mounted on the periphery of a boundary B wherein current is injected in pairs of electrodes at the perimeter or boundary B and voltages are read at all other electrodes E. The position of the readings is then rotated and the readings are repeated. An open source algorithm EIDORS reconstructs conductivity change at points within the sensor 130.

The boundary voltage BV is an inverse problem wherein the conductivity distribution of the electrical material is analogous to tactile sensing of force and strain.

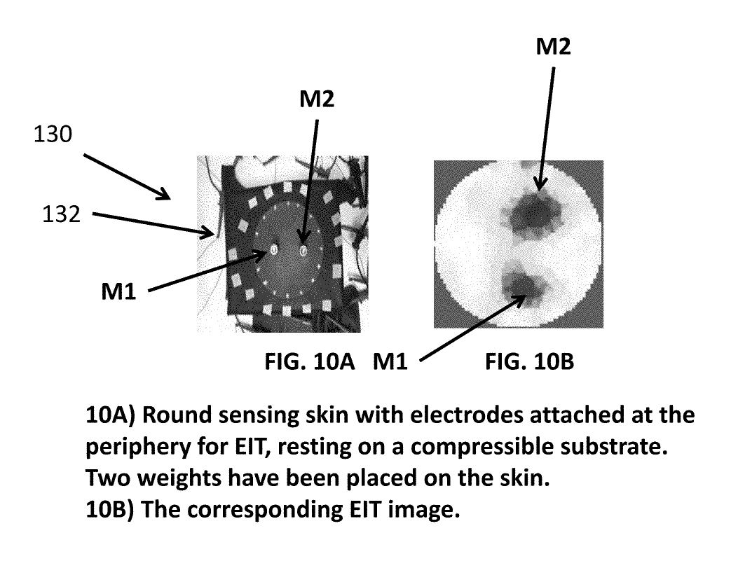

FIG. 9 illustrates the sensor 130 in a distributed system 140 which now includes Multiplexers MP1 and MP2 wherein analog input measurements are transmitted to a data acquisition card (DAQ) 320 where the analog input measurements are converted to digital output.

FIG. 9A1 illustrates two loading points M1 and M2 for a mechanical sensor diameter of 10 cm where the electrical reading images are shown as dark spots M1 and M2 in FIG. 9A2.

FIG. 9B1 illustrates a thermal sensor 135 having a square outline boundary and wherein thermal sensing readings are shown as a quadrilateral image M′ in FIG. 9B2.

This illustrates the advantages of the mechanical sensor readings utilizing the distributed system 140 as compared to the thermal sensor readings.

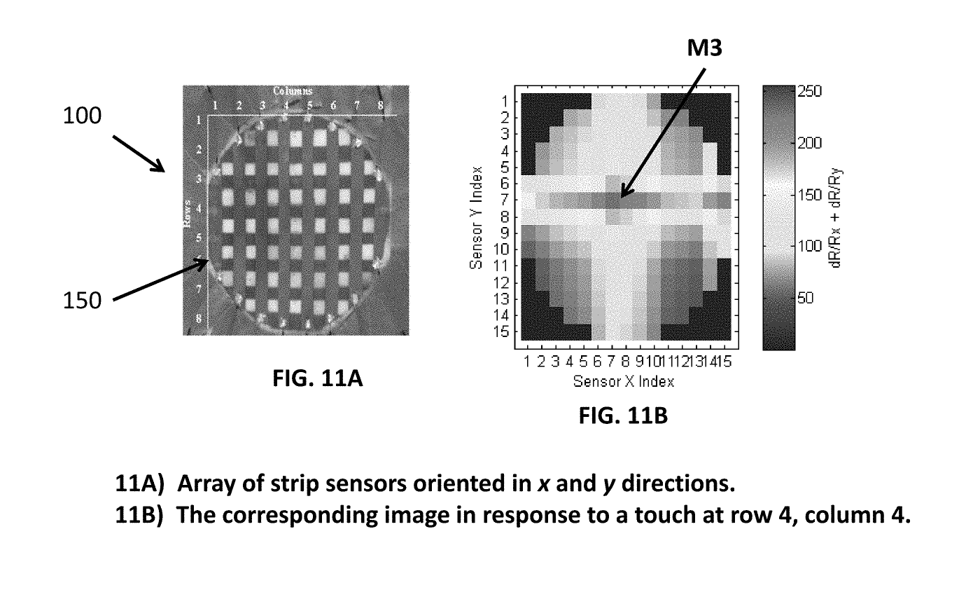

FIG. 10A illustrates a detailed view of the electrical sensor 130 with electrodes E attached at the periphery for EIT and resting on a compressible substrate 132.

FIG. 10B illustrates the corresponding EIT image showing the dark areas M1 and M2 represented tumor locations.

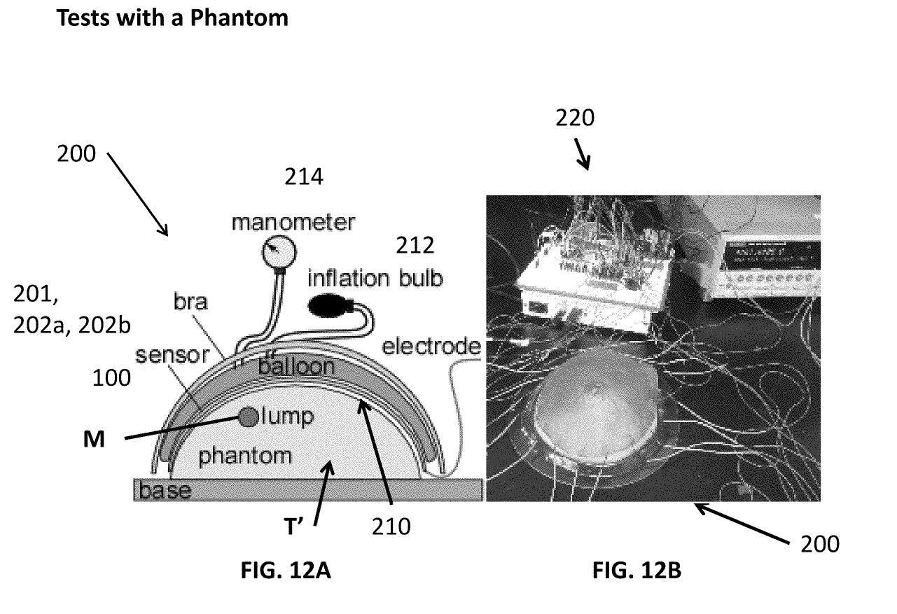

FIG. 11A illustrates an array strip sensor 150 that is formed of a series of orthogonally positioned crossing strips of eight (8) rows and eight (8) columns.

FIG. 11B illustrates the corresponding image in response to a touch at row 4, column 4.

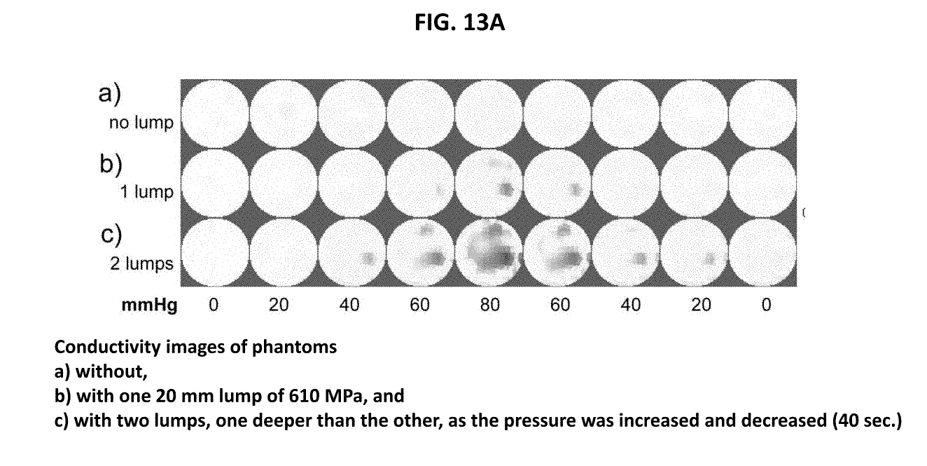

FIG. 12A illustrates the pressure sensing system 100 configured as a tumor detection system 200 as described above with respect to FIGS. 7A-7C but as a phantom for testing.

FIG. 12B illustrates portable electronics system 220 that is in electrical communication with the tumor detection system 200.

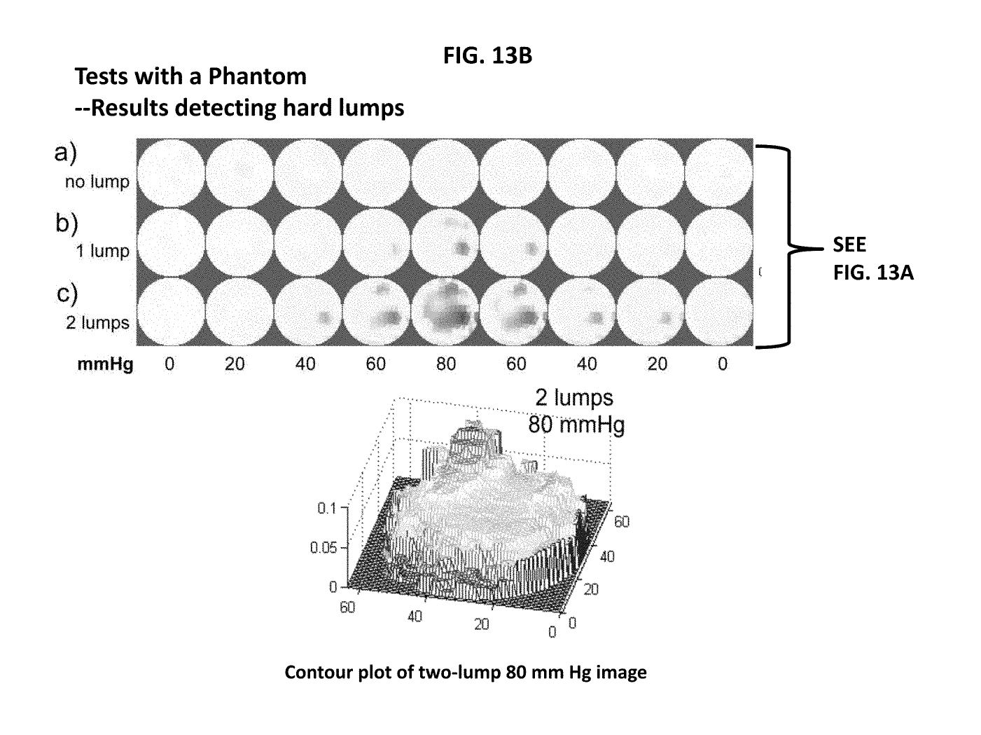

FIG. 13A illustrates conductivity images converted to mm Hg of phantoms (a) with no lumps; (b) with one (1) lump; and (c) with two (2) lumps.

FIG. 13B illustrates a contour plot of the two-lump 80 mm Hg image.

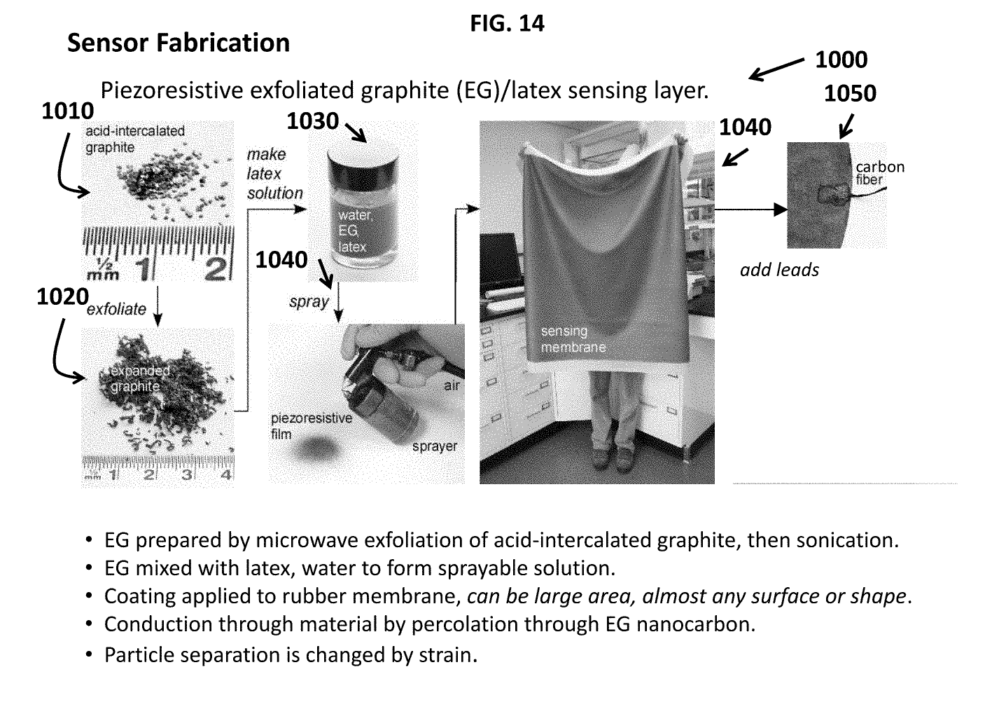

FIG. 14 is a schematic diagram for a method 1000 of manufacturing the piezoelectric exfoliated graphite (EG)/latex sensing layer which includes in step 1010 preparing the piezoelectric exfoliated graphite (EG)/latex sensing layer by microwave exfoliation of acid-intercalated graphite.

Step 1020 includes sonicating the piezoelectric exfoliated graphite (EG)/latex sensing layer that has been prepared in step 1010 by microwave exfoliation of acid-intercalated graphite.

Step 1030 includes mixing the piezoelectric exfoliated graphite (EG)/latex sensing layer with latex and water to form a sprayable solution.

Step 1040 includes spraying the sprayable solution to a rubber membrane. The rubber membrane may be formed in a large area and generally unrestricted in surface or shape.

Step 1050 is shown as part of the manufacturing process but relates to application of the EG to the pressure sensing, i.e., conduction through the piezoelectric exfoliated graphite (EG)/latex sensing layer occurs by percolation through EG nanocarbon. Particle separation is changed by strain.

While several embodiments and methodologies of the present disclosure have been described and shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular embodiments and methodologies. Those skilled in the art will envision other modifications within the scope of the claims appended hereto.

What is claimed is:

1. A pressure sensing system comprising:
at least two pressure sensing layers,
a first pressure sensing layer of the at least two pressure sensing layers including:
a first sensing system configured in a layer; and
a first layer of foam having a Young's Modulus and mounted between the first sensing system configured in a layer and a second sensing system configured in a layer; and
at least a second pressure sensing layer of the at least two pressure sensing layers including:
the second sensing system configured in a layer; and
a second layer of foam having a Young's modulus that is greater than the Young's modulus of the first layer of foam and mounted between the second sensing system configured in a layer and a rigid substrate having a Young's modulus greater than the layer of the first sensing system, the first layer of foam, the layer of the second sensing system, and the second layer of foam,
the at least two pressure sensing layers defining thereby a multi-layer pressure sensing system.

2. The pressure sensing system according to claim 1, wherein the multi-layer pressure sensing system includes an electrical impedance tomography circuit.

3. The pressure sensing system according to claim 2, wherein the electrical impedance tomography circuit includes a plurality of pairs of adjacent electrodes, wherein current is injected into an adjacent pair of electrodes such that voltage readings obtained from the remaining pairs of the plurality of pairs of adjacent electrodes enable reconstruction of an image from the voltage readings.

4. The pressure sensing system according to claim 3, wherein the electrical impedance tomography circuit includes circuitry enabling wireless transmission of the voltage readings and current data readings from the multi-layer pressure sensing system to a remote receiver location.

5. The pressure sensing system according to claim 1, wherein the multi-layer pressure sensing system includes an array of strip sensors disposed over a layer of foam padding.

6. The pressure sensing system according to claim 1, wherein the pressure sensing system is configured as a tumor detection system, the tumor detection system including:
an anatomical contact material configured to contact or apply pressure to at least one anatomical mass that extends from a body of a user of the system or to a body surface of a user of the system,
the anatomical mass including an outer surface with respect to the body of the user,
the anatomical contact material including an interior surface and an exterior surface with respect to the outer surface of the at least one anatomical mass or to the body surface,
the multi-layer pressure sensing system including an interior surface and an exterior surface with respect to the outer surface of the at least one anatomical mass or to the body surface,
the interior surface of the multi-layer pressure sensing system configured to be positioned over the outer surface of the at least one anatomical mass, or body surface, between the at least one anatomical mass, or body surface, and the interior surface of the anatomical contact material,
wherein the rigid substrate of the multi-layer pressure sensing system is configured as a flexible insufflation reservoir including an interior surface and an exterior surface with respect to the outer surface of the at least one anatomical mass or the body surface,
the flexible insufflation reservoir configured wherein the interior surface of the flexible insufflation reservoir can be positioned over the exterior surface of the multi-layer pressure sensing system and wherein the interior surface of the anatomical contact material can be positioned over the exterior surface of the flexible insufflation reservoir,
wherein inflation of the flexible insufflation reservoir causes pressure to be applied to the multi-layer pressure sensing system and to the at least one anatomical mass, or body surface, to enable detection of a tumor within the at least one anatomical mass, or body surface, by the multi-layer pressure sensing system.

7. The pressure sensing system according to claim 6, wherein the multi-layer pressure sensing system includes an electrical impedance tomography circuit.

8. The pressure sensing system according to claim 7, wherein the electrical impedance tomography circuit includes a plurality of pairs of adjacent electrodes, wherein current is injected into an adjacent pair of electrodes such that voltage readings obtained from the remaining pairs of the plurality of pairs of adjacent electrodes enable reconstruction of an image from the voltage readings.

9. The pressure sensing system according to claim 8, wherein the electrical impedance tomography circuit includes circuitry enabling wireless transmission of the voltage readings and current data readings from the multi-layer pressure sensing system to a remote receiver location.

10. The pressure sensing system according to claim 6, wherein the multi-layer pressure sensing system includes an array of strip sensors disposed over a layer of foam padding.

11. The pressure sensing system according to claim 6, wherein the anatomical contact material configured to contact at least one anatomical mass that extends from the body of a user is configured as a brassiere to contact breasts of a user to detect tumors occurring within at least one breast of the user.

12. The pressure sensing system according to claim 6, wherein the anatomical contact material configured to contact at least one anatomical mass that extends from the body of a user is configured as a male athletic supporter to contact testicles of a male user to detect tumors occurring within at least one testicle of the male user.
